(19) United States
(12) Patent Application Publication
(10) Pub. No.: US 2004/0035421 A1
(43) Pub. Date: Feb. 26, 2004

(54) Dosing device which can be activated by suction air stream of the user

(76) Inventor: Alfred Von Schuckmann, Kevelaer (DE)

Correspondence Address: Martin A Farber, Suite 473, 866 United Nations Plaza, New York, NY, 10017, US

(21) Appl. No.: 10/381,541

(22) PCT Filed: Sep. 14, 2001
(86) PCT No.: PCT/EP01/10651

(30) Foreign Application Priority Data
Sep. 27, 2000 (DE) 100 47 722.4

(51) Int. Cl. A61M 16/10; A61M 15/00; A61M 16/00

(52) U.S. Cl. 128/203.15; 128/203.12

(57) ABSTRACT

Dosing device (D) which can be activated by stream (s) of suction air of a user and is intended for dispensing from a powder supply in a supply chamber (2) reproducible sub-quantities (1′ of powder (1), in particular powdered medicament, which is to be inhaled by the user, wherein a sub-quantity (1′) located in a dosing chamber (16), which has a dosing-chamber base (34) and a dosing-chamber wall (35), in a first instance is to be moved manually into a ready-to-dispense position and thereafter a sucking action which overcomes a threshold value activation takes place for transferring the sub-quantity (1′) into the suction air stream (S), further comprising a suction-air-controlled displacement of a displaceable sub-region of the dosing chamber (16) for exposing the sub-quantity (1′) to the suction air during the sucking action which exceeds a threshold value.

The invention relates to a dosing device which can be activated by the suction air stream of the user and is intended for dispensing from a supply chamber reproducible sub-quantities of powder, in particular powdered medicament, which is to be inhaled by the user, it being the case that a sub-quantity located in a dosing chamber, which has a dosing-chamber base and a dosing-chamber wall, in the first instance is to be moved manually into a ready-to-dispense position and then a sucking action which overcomes a threshold value activation takes place for transferring the sub-quantity into the suction air stream.

Propellant-free inhalers in which the action of clearing out the cavity-like dosing chamber which accommodates the sub-quantity is based on the force of the suction air stream of the user are common. This force may differ greatly from case to case, so that it is not always ensured that the powder is carried to the target area (bronchial tubes, lungs). In any case, it is quite possible for a number of aborted attempts to precede eventual success. This may be observed, in particular, in the case of users with shallow breathing. Acceptance of such dosing devices is thus lacking.

EP patent specification 0 549 605 proposes to add extraneous air. The user's suction air up to a threshold value is used here for freeing a quantity of air which, for all practical purposes, is kept in a store. The threshold value may be set at a fairly low level here. The quantity of air is located in a pump piston/cylinder unit. The spring-stressed, piston-carrying pump shank is triggered, this generating a compressed-air stream, via a ratchet-like switching device with a disengageable safety catch, this device reacting to the negative pressure produced when the user breathes in. The means involve relatively high outlay. The compressed-air stream empties the dosing chamber, freeing the sub-quantity taken up. The air stream passes through the dosing chamber. The powder, passing through a nozzle located in the mouthpiece, is thus blown out and dispersed. The sub-quantity is taken up by the batch of powder being discharged vertically downward from a hopper-like supply chamber or by radial supply via a lateral, recloseable window of the dosing chamber. A screen which partitions off the latter divides the dosing chamber from the incoming compressed-air flow path and holds the sub-quantity together against dropping out into the air supply. The introduction of the extraneous air stream into the respiratory suction air stream at the very least takes some getting used to.

It is an object of the invention to form a dosing device of the generic type in a structurally simplified manner such that activation for transferring the sub-quantity takes place in a functionally reliable manner without the addition of extraneous air.

This object is achieved first and foremost, in the case of a dosing device or an inhaler having the features of claim 1, by a suction-air-controlled displacement of a sub-region of the dosing chamber for exposing the sub-quantity to the suction air during the sucking action exceeding the threshold value. The divided-off quantity is then positioned in the suction air stream and is no longer blown out by a separate device which generates compressed air. The transporting means, air, is kept at a negative pressure in accordance with the sucking action. It lies within the range of the normal suction force of the user for a partial force to be set aside as dispenser. The result is complete discharge of the substance positioned in the suction air stream. The dosing chamber, as it were, comes apart. Only a sub-region of the dosing chamber is displaced for this purpose. This can easily be managed in mechanical terms and can also be achieved with a low level of friction. It is advantageous here for the displaceable sub-region to be in the form of a negative-pressure-controlled dosing-chamber base which can be raised relative to the dosing-chamber wall. The dosing-chamber wall draws back behind the dosing-chamber base. The dosing-chamber wall achieves its ready-to-dispense position beforehand by manual operation. Furthermore, it is an advantageous feature of the invention that the dosing chamber is in the form of a tube which accommodates the movable dosing-chamber base and can be moved with scooping action through the powder supply. With scooping action means starting from beneath the supply chamber, and in this sense may even likewise be referred to as a raising action. The said ready-to-dispense position may be achieved manually by straightforward means in that the dosing chamber is coupled to a closure cap of the dosing device and, in the course of the closure cap being removed from a housing of the dosing device, is moved with scooping action through the powder supply. The ready-to-dispense position is thus brought about automatically and is also correspondingly eliminated again following application of the sub-quantity and correct closure of the inhaler or of the dosing device. The means for corresponding actuation are straightforward and expedient. The procedure here is such that the closure cap is coupled in a releasable manner by means of clip-action claws to a connecting part which carries the dosing chamber. The connecting part functions as a carrier of the tube, which has a guiding action in relation to the dosing-chamber base. The claws act in the manner of push rods when the dosing device is closed and in the manner of carry-along claws when the dosing device is opened. The dosing-chamber base, which can be moved relative to the dosing-chamber wall, is coupled to the tube here via driver protuberances. The latter keep the dosing-chamber base spaced apart from the supply-chamber-side tube edge by a scoop-producing distance, forming an axial displacement, which is sufficient for the abovedescribed action of exposing the sub-quantity to the suction air. Furthermore, it is provided that the dosing-chamber base is acted upon by a spindle, that end of the latter which is directed away from the supply chamber having a pressure-exerting base actuated by suction air stream. Furthermore, it is beneficial that the dosing-chamber base can be adjusted relative to the spindle in order to achieve different sub-quantities. This is embodied in straightforward structural terms by a screwing-action adjustment capability of the dosing-chamber base relative to the spindle. Stepless adjustment of the volume of the dosing chamber is possible. In respect of the reproducible dividing accuracy which is to be sought here, it also proves to be advantageous for the supply chamber to contain a scraper which interacts with the dosing chamber as it passes through. A structurally straightforward solution is provided here if the scraper is in the form of a radial finger which can be pushed aside from the displacement path. As a development, the invention proposes the feature of a supply-chamber ceiling being in the form of a membrane, which allows through-passage as a result of slitting, or the ceiling being assigned such a membrane. The slitting here is expediently a cross-slit arrangement, so that identically sized lugs are provided which can be flapped out of the way and position themselves against the tube wall, forming a through-passage in the process, as a result of their inherent restoring force. In this context, it is beneficial and provided that the length of a slit is adapted to the dosing chamber passing through it such that the exiting dosing chamber is enclosed in a sealing manner by the membrane. The foot region of the lugs produces, as it were, a protruding, not just sealing, but also guiding, annular lip. In a dosing device which has a mouth aperture which is located above the supply chamber and around which the user is to place his/her mouth, it is advantageous, in respect of a good swirling action of the pulverulent substance, for a flow divider to be disposed in the mouth aperture. This flow divider is expediently realized as a central body which tapers cross-sectionally in the direction of the dosing chamber. It is then proposed that the closure cap is accommodated in the housing, which retains the dosing chamber, via a screw thread.

The subject matter of the invention is explained in more detail hereinbelow with reference to an exemplary embodiment illustrated in the drawings, in which:

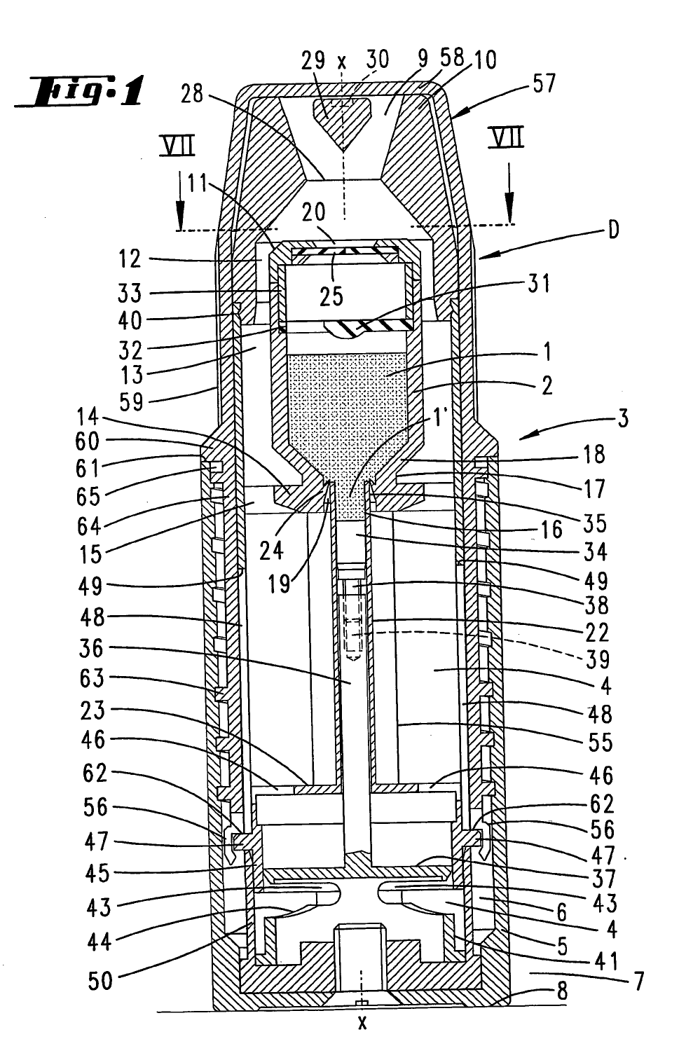

FIG. 1 shows the dosing device, which is realized as an inhaler, in vertical section, to be precise in the basic position, in which it is closed by a closure cap,

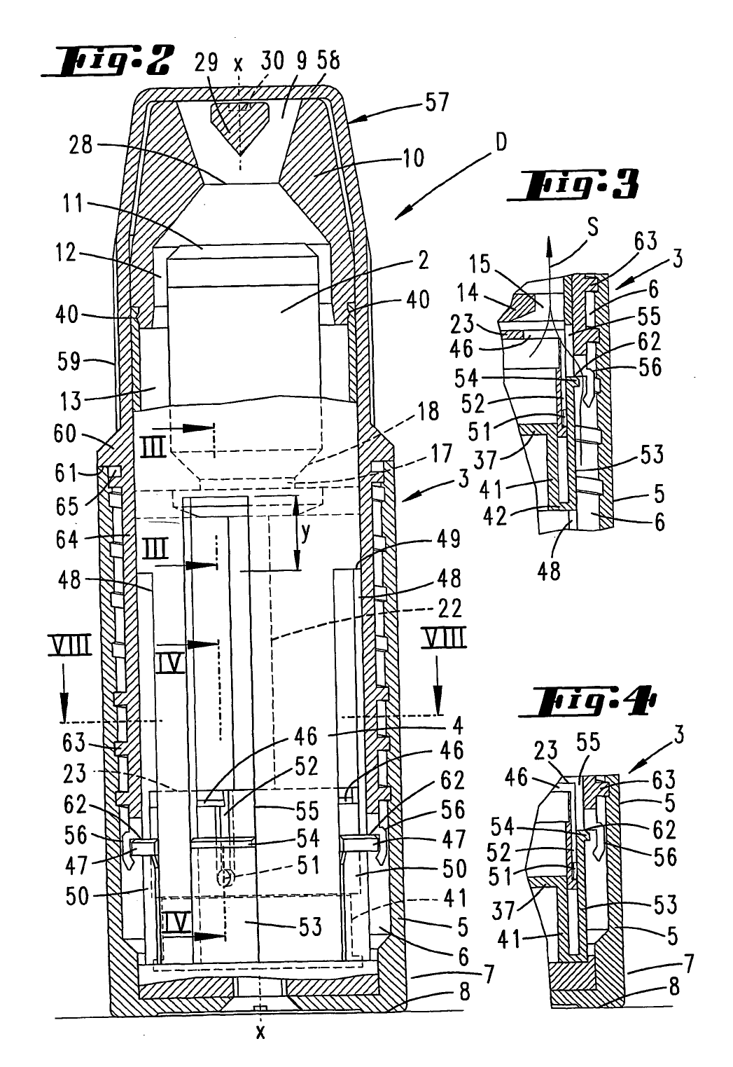

FIG. 2 shows the dosing device in the same position as FIG. 1, in a partially sectional illustration (the core region has been represented in a predominantly non-sectional manner),

FIG. 3 shows the section along line III-III in FIG. 2,

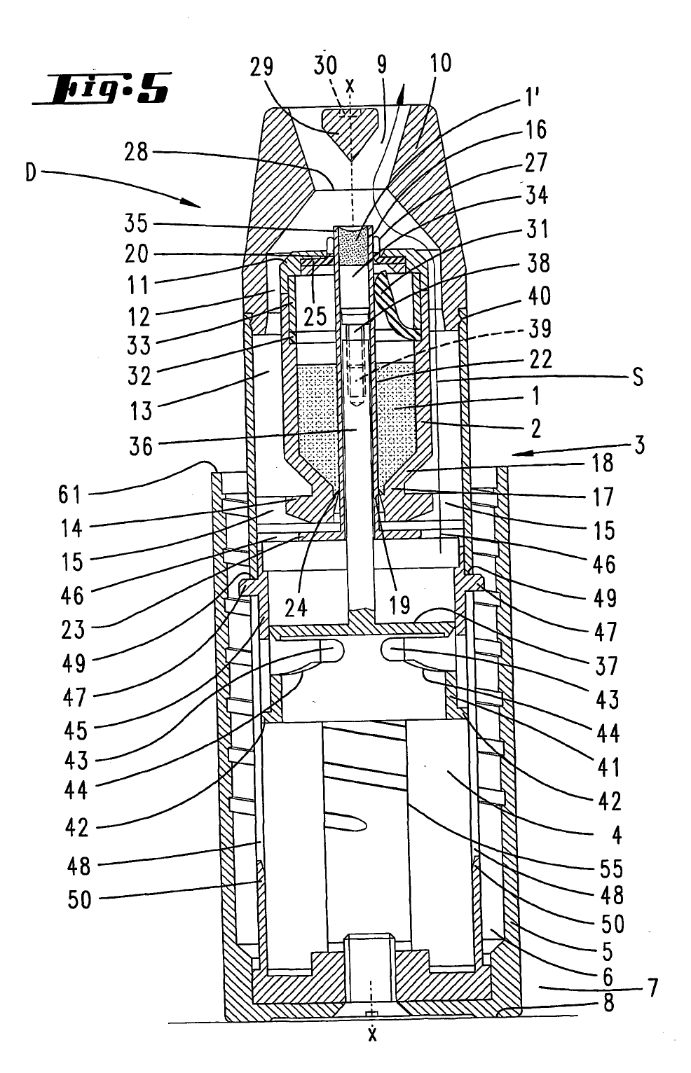

FIG. 4 shows the section along line IV-IV in FIG. 2,

FIG. 5 shows the dosing device in vertical section, to be precise in the manually brought-about ready-to-dispense position and with the closure cap correspondingly removed, this giving rise to the opening-movement-dependent displacement of the dosing chamber,

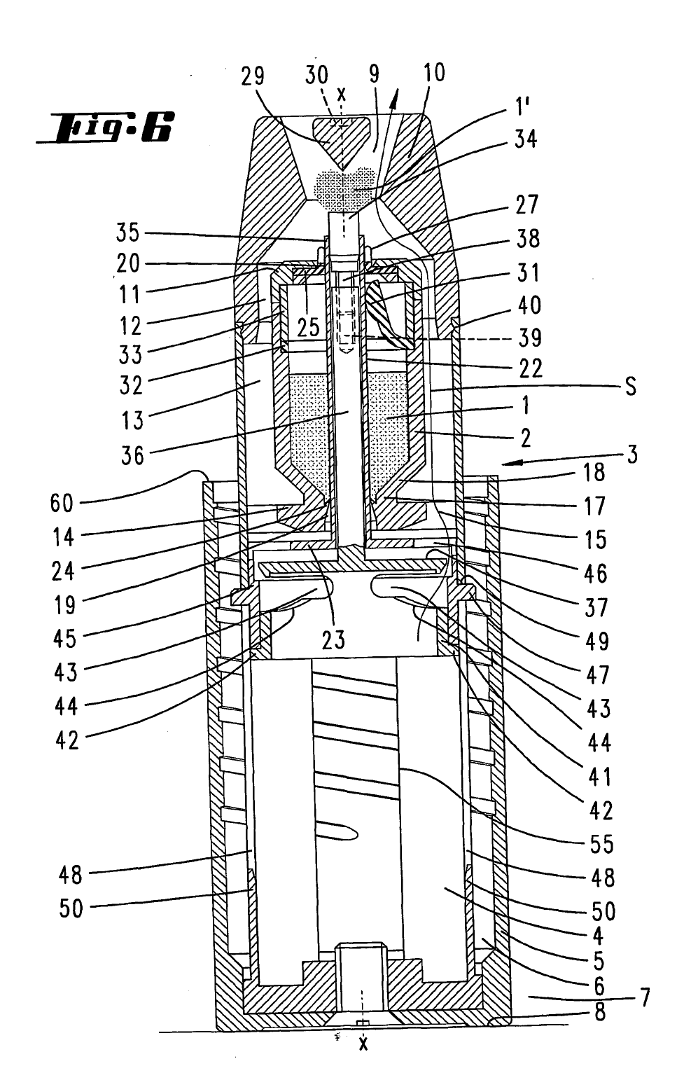

FIG. 6 shows the same section as FIG. 5, with a sub-quantity of powder discharged to the suction air stream by sucking action,

FIG. 7 shows the section along line VII-VII in FIG. 1,

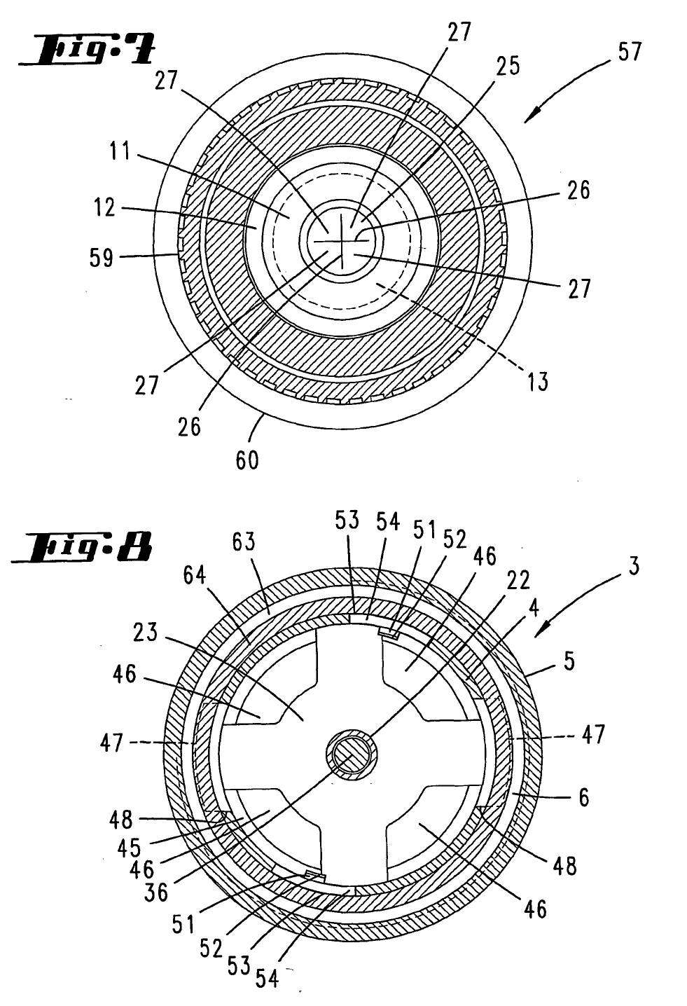

FIG. 8 shows the section along line VIII-VIII in FIG. 2,

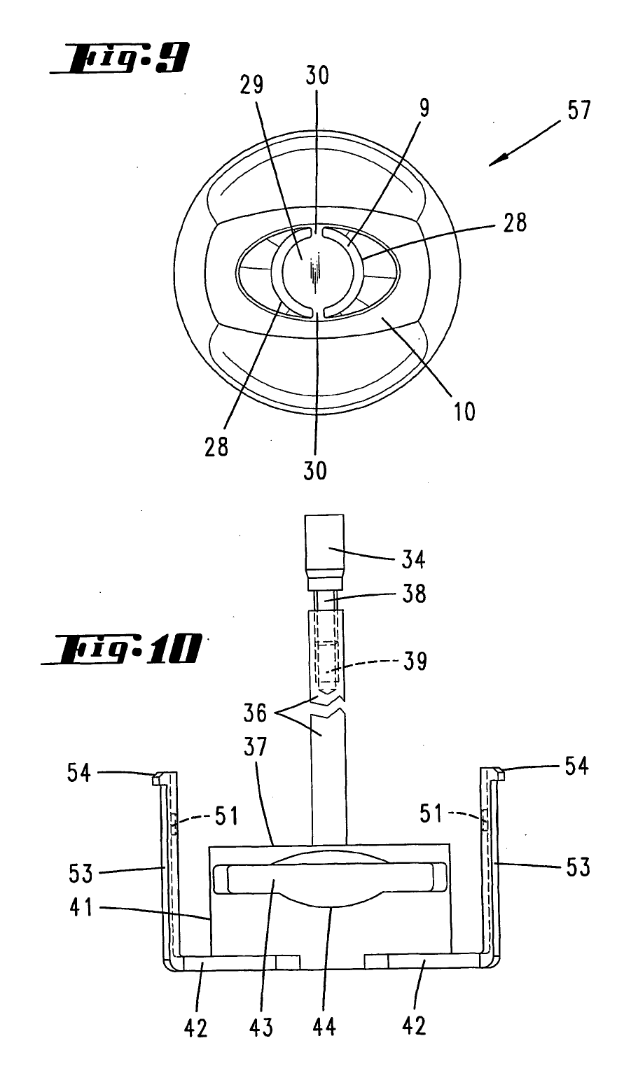

FIG. 9 shows a plan view of the mouthpiece of the dosing device, and

FIG. 10 shows a side view of the spindle, with associated pressure-exerting base, illustrated on its own.

The dosing device D, which serves as an inhaler, makes it possible to dispense reproducible sub-quantities 1′ of a powder 1, preferably powdered medicaments.

The powder 1 is contained in a supply chamber 2 of the dosing device D such that it may or, in the case of a disposable version, may not be refilled. The filling capacity of the said supply chamber corresponds to the multiple of a sub-quantity 1′.

The predominantly cylindrical supply chamber 2 is accommodated in a double-walled housing 3. This comprises an inner annular wall 4 and an outer annular wall 5, with a gap 6 between the two. This gap extends, at one end of the dosing device D, as far as a base 7 of the housing 3. This base forms at its periphery a standing edge 8, so that the dosing device D, which can be carried along in the pocket, may be set down, for example, in order for powder 1 to be refilled.

The inner annular wall 4 is somewhat longer than the outer annular wall 5.

Opposite the closed base 7, the dosing device D continues at the other end, that is to say at the top of the drawing, into an open mouth aperture 9, which is formed in a mouthpiece 10 clipped to the outer annular wall 5. The corresponding arrangement may be reversible and thus, by virtue of the entire mouthpiece 10 being drawn off, allows access to a then exposed ceiling 11, which keeps the supply chamber 2 closed at the top.

The mouth aperture 9 continues into a suction channel 12 encircling the supply chamber 2. This suction channel is continued in the interior 13 of the housing 3 and, finally, is connected to the outside air via the gap 6.

The wall of the supply chamber 2 is rooted in a transverse wall 14 of the housing 3. This extends just beneath the upper end of the outer, shorter annular wall 5 and is interrupted around the periphery. There are four arcuate slots which are interrupted by crosspieces and have a beveled inner edge. The interruptions are designated 15. The suction channel 12 continues, via these, in the direction of the base 7.

The sub-quantity 1′ is measured and discharged from the supply chamber 2 via a dosing chamber 16. This takes place in the manner of an elevator by the sub-quantity 1′ being raised from the mass of powder illustrated, to be precise with scooping action. The mass of powder loosens at the same time and avoids blockage of the powder substance.

In the basic position (FIG. 1) of the dosing device D, the dosing chamber 16 is located on the base 17 of the supply chamber 2. The base 17 opens out into the transverse wall 14 on the upper side. On the mouth-aperture side, it merges into a hopper 18 of the wall of the supply chamber 2. The hopper widens in this direction.

The base 17 has a central through-opening 19.

A similar central through-opening 20, congruent to the through-opening 19, is also located in the supply-chamber ceiling 11. The two are spaced apart axially along a common longitudinal center axis x-x of the substantially rotationally symmetrical inhaler.

The through-openings 19, 20 are coordinated with the external diameter of a dosing chamber 16. The latter, in the basic position, is plugged in the through-opening 19, thus filling the hole in a stopper-like manner. It is not possible to go below this height position.

The wall of the dosing chamber continues into an elongate tube 22. This extends from a connecting part 23 which functions as a carrier. The connecting part 23 is guided in the interior of the inner annular wall 4. The cylindrical tube 22 is of such a length that, in the ready-to-dispense position of the dosing device D which can be seen from FIG. 5, it projects through the entire interior of the supply chamber 2, to be precise in a sealed manner both in the region of the through-opening 19 remote from the mouth aperture and of the through-opening 20 in the vicinity of the mouth aperture.

The means used for sealing purposes in relation to the through-opening 19 remote from the mouth aperture is a sealing lip 24 which projects from the hole-forming annular wall of the transverse wall 14 at this location. The sealing lip is integrally formed and occupies the narrowest point of the hopper 18. Its rotationally symmetrical convergence to the inside diameter of the through-opening 19 allows the powder 1 to be dispensed from the supply chamber 2 without any residues being left. The projecting sealing lip 24 tapers in the direction of the supply chamber 2. The seal in the region of the through-opening 20 in the vicinity of the mouth aperture comprises a membrane 25 which is made of elastomeric material and is associated with the supply-chamber ceiling 11 or is integrally formed therewith. The said membrane 25, which lines the through-opening 20 in a sealing manner, has a slit arrangement. As FIG. 7 shows, this is a cross-slit arrangement. The slits 26 intersect in the center, that is to say at the longitudinal center axis x-x of the dosing device D.

The slits 26 are of such a length as to produce an opening which allows the pushing-up dosing chamber 16 to pass through, the ceiling being opened in the process. The lateral wall of the dosing chamber is enclosed in a sealing manner by the lugs 27 produced by the cross-slit arrangement. The length of the slits 26 of the membrane 25 even leaves behind a closed root as a supporting bead, so that it is not possible for any powder 1 to pass into the region of the mechanics of the dispenser. On the other hand, however, the swung-out position of the lugs 27 is stable enough for the lugs 27 not to be turned back when the dosing chamber 16 moves back.

That portion of the mouth aperture 9 which adjoins the supply-chamber ceiling 11 tapers to a constriction 28. From this constriction 28, the mouth aperture 9 changes in the outward direction again into a significantly widened portion. A flow divider 29 is accommodated in this part of the mouth aperture 9. The flow divider is realized as a central body which tapers cross-sectionally in the direction of the dosing chamber 2. The central body is connected to the inner wall of the widened portion or mouth aperture 9 via radial crosspieces 30. The flow divider 29, which establishes a good swirling action of the pulverulent substance which is to be dispensed, is realized in specific terms, in respect of its actively dividing part, as a cone. Its tip is located within the longitudinal center axis x-x. The base diameter of the cone is less than the inside diameter of the constriction 28 (see FIG. 9).

The dosing chamber 16, which raises the sub-quantity 1′, is assigned a filling-limiting means. This ensures precisely reproducible doses despite the possible inclination of the powder 1, causing the latter to be piled up over the edge of the, as it were, cup-like dosing chamber 16. For this purpose, the powder-free, upper portion of the supply chamber 2 is assigned a scraper 31. In the basic position, this extends parallel to the transverse wall 14 and supply-chamber ceiling 11. It crosses over the displacement path of the dosing chamber 16 by way of its inner, free end, which is shaped into a dome on the underside, see FIG. 1. When the supply chamber 16 is displaced into the ready-to-dispense position according to FIG. 5, the scraper 31, which is configured as a radial finger, is pushed aside from the displacement path, carrying out the scraping operation in the process. This situation can be gathered from FIG. 5, which also shows the indent-like impression on the sub-quantity 1′ which is correspondingly produced by the dome shape.

The radial finger or scraper 31 is rooted at the periphery in a ring 32. The latter is supported on a shoulder of the wall of the supply chamber 2, this shoulder being produced by the wall being offset, and is secured on the upper side by a sleeve 33 which extends from the underside of the supply-chamber ceiling 11, which is formed, for example, as a hard part. The sleeve 33 may serve, at the same time, as a plug-protrusion for the then lid-like supply-chamber ceiling 11. They are connected with a friction fit. The unit 11/33 may also be a screw top, with a mating thread on the supply chamber 2.

The lower termination of the cup-like dosing chamber 16 is formed by a dosing-chamber base 34. The dosing-chamber wall 35 normally projects beyond the dosing-chamber base, this dosing-chamber wall encircling the dosing chamber 16 and, as has been said, being formed by a portion of the tube 22. The dosing-chamber base 34 can be moved, and also adjusted, relative to the dosing-chamber wall 35 in order to change the volume of the dosing chamber 16.

Its (35) operationally induced movement is utilized for emptying the dosing chamber 16 by the sub-quantity 1′ being transferred into the suction air stream S. For supplying the suction air stream S, the user places his/her mouth around the mouth aperture 9. The mouthpiece 10 has a flattened portion which is appropriate for the mouth (see FIG. 9).

The volume-changing adjustment in order to obtain different sub-quantities 1′ can be achieved by screwing action. The dosing-chamber base 34 may be correspondingly adjusted by screwing action relative to a spindle 36. The latter acts upon the dosing-chamber base 34.

The movement-transmission means of the spindle 36 is a pressure-exerting base 37. This is located at that end of the spindle 36 which is directed away from the supply chamber 2.

The pressure-exerting base 37 is located in the flow region or region of action of the suction air stream S.

For the screwing-action capability of the dosing-chamber base 34, the latter has a threaded pin 38 on the spindle side. The external thread thereof interacts with the internal thread of a centrally located blind bore 39 of the spindle 36. The adjustment capability is stepless and is effected in the emptying position according to FIG. 6, in which part of the dosing-chamber base 34 projects freely from the dosing-chamber-forming tube end at this location. Accessibility is achieved via the above-explained removability of the mouthpiece 10, the offset bottom edge of which carries clip-action means 40 in the direction of the end of the inner annular wall 4 at this location.

The pressure-exerting base 37 merges, remote from the supply chamber 2, into a cylindrical cup wall 41. This terminates at the end into an outwardly directed guide edge 42.

Just beneath the disk-like pressure-exerting base 37, the cup wall 41 has window slots 43 located at the same height. These are widened axially by a convex niche 44 on the guide-edge side. On the pressure-exerting-base side, the niche 44 is located opposite a bevel (see FIG. 10).

While the projecting guide edge 42 slides on the inside of the inner annular wall 4, the periphery of the pressure-exerting base 37 is guided in the interior of a cup wall 45 of the connecting part 23.

The lateral section of the cup wall 45 is of cylindrical configuration and along the final third toward the disk-like connecting part 23, that is to say the cup base, is configured with a greater cross section, so that flow can take place around the periphery of the pressure-exerting base 37, that is to say the periphery of the pressure-exerting base is in connection with the suction channel 12. This is achieved by a reduction of the cup wall 45 at this location. The connection to the suction channel 12 is formed by openings 46 in the base of the cup-like connecting part 23. These openings are arcuate through-openings. They are interrupted by radial material bridges, see FIG. 8. The openings 46 also go as far as the narrowed wall extension of the cup wall 45 and extend, for all practical purposes, opposite the through-openings 15 of the transverse wall 14.

Driver protrusions 47 extend radially outward from the lateral cup wall 45 of the connecting part 23. These driver protrusions are located diametrically opposite one another and engage through axially oriented longitudinal slots 48 of the inner annular wall 4 of the housing 3. The width of the driver protrusions 47 corresponds, with a slight amount of play, to the width of the longitudinal slots 48. This results in the connecting part 23 being guided in a rotationally secured manner.

In addition to the driver function of the driver protrusions 47, the latter are also used for an end-stop function. Accordingly, their upper side passes against the upper end of the longitudinal slot 48, forming a stop 49. With the connecting part 23 located in the basic position, the underside comes up against the corresponding end side of a stop lug 50 extending from the base 7.

The guided axial movement of the parts 23 and 37 in relation to one another is likewise defined by end stops. For this purpose, use is made, on the one hand, of the guide wall 42 of the pressure-exerting base 37 and, on the other hand, of the lower end edge of the cup wall 45 of the connecting part 23. This stop situation can be gathered from FIG. 6. It corresponds to the situation where the dosing chamber 16 is cleared out. The chamber-forming situation, shown for example in FIG. 5, the so-called ready-to-dispense position, is based, in control terms, on stop-creating means such as driver protuberances 51. These are seated on the part which forms the pressure-exerting base 37, see FIGS. 3 and 4. These likewise diametrically opposite driver protuberances 51 are directed radially inward and run in an axial guide slot 52 in the lateral section of the cup wall 45 of the connecting part 23. The flat-cylindrical driver protuberance 51 is stopped by that end of the guide slot 52 which is in the vicinity of the base. The guide slot 52 is connected to the openings 46 so as to allow throughflow.

The axially oriented displacement travel between the parts 23 and 37 is dimensioned such that the driver protuberance 51 cannot pass out of the end of the guide slot 52, which is left open at the top.

The driver protuberance 51 is seated on an axially oriented strip 53. This extends from the periphery of the guide edge 42 of the pressure-exerting base 37. It projects beyond the upper side of the same to a considerable extent and merges into a strip head 54. This projects radially outward, forming a protrusion in the process. The strips 53, which are realized diametrically opposite one another in pairs, are likewise guided in each case in a longitudinal slot 55 of the inner annular wall 4 of the housing 3. Here too, use is made of a width formation which leaves a corresponding amount of play.

Both the driver protrusions 47 and the strip heads 54 project into the gap 6 of the housing 3. They are thus located in the region of action of claws 56 of an actuating device, with the aid of which the filled dosing chamber 16 is moved into a ready-to-dispense position which is brought about automatically by hand. The basis of such an actuating device is a closure cap 57, which is made up of an actual conical cap 58, which closes the mouth aperture 9 of the dosing device D, and a more cylindrical actuating portion 59, the lateral wall of which is roughened, and in specific terms has longitudinal grooves. It has a stop collar 60 engaging over the gap 6. This stop collar comes up against the corresponding end surface of the outer annular wall 5 of the housing 3, forming a counter stop 61. The stop situation here is such that the connecting part 23 and the pressure-exerting base 37 are located in the basic position, which can be seen from FIG. 1. This is achieved via the claws 56 explained above. The abutment flank 62 of the latter, this flank being located perpendicularly to the longitudinal center axis x-x, engages against the respective upper side of the driver protrusions 47 and strip heads 54.

The corresponding downward displacement takes place in the course of a rotary movement. This is converted, as a result of a screw thread 63, into a downwardly directed axial movement. The screw thread 63 is located on a sleeve-like continuation 64 of the actuating portion 59 of the closure cap 57. It is realized thereon as an external thread which engages in a matching internal thread on the inside of the outer annular wall 5. A coarse thread is preferred.

At the inner free end of the sleeve-like continuation 64, the claws 56 are seated in a row along a circle line around x-x. They are separated from one another by tongue-forming slots extending from the lower edge. The slot spacing is such that good resilient deflection is present, use also being made here of the circular curvature. Such resilient deflection is used in relation to the driver protrusions 47, since the claws 56 only grip over the latter in a latching manner. The corresponding clip-action engagement of the claws 56 is eliminated when the closure cap 57 is unscrewed. The position according to FIG. 5 is then present. Accordingly, the dosing chamber 16 is coupled to the closing cap 57 of the dosing device D such that, in the course of the closure cap 57 being removed from the housing 3 of the dosing device D, the dosing chamber 16 is moved with scooping action through the powder supply. This administering position is maintained with a friction fit. The pressure-exerting base 37 here is carried along not by the claws 56, but via the driver protuberances 51 and the stop-forming end of the guide slot 52 of the connecting part 23. The fact that the strip head 54 is not located in the latching carry-along region of action of the hook-like, resilient claw 56 can be gathered from FIGS. 3 and 4.

For the latching engagement of the hook-like claw 56 over the driver protrusions 47, which project somewhat further outward, is assisted by sloping run-over flanks of the claws 56 and by an edge bevel on the upper side of the driver protrusions 47. The underside of the hook mouth also runs in a sloping manner, so that the claws 56 can slide off for uncoupling purposes.

In order to cover the additional travel which positions the sub-quantity 1′ in the suction air stream S, the longitudinal slots 55 which guide the strip heads 54 are longer on the mouth-aperture side of the dosing device D. The corresponding extent of travel is depicted in FIG. 2 and designated y. It covers a displacement length of the dosing-chamber base 34 which not only empties the volume of the dosing chamber 16, but raises the sub-quantity 1′ over the edge of the chamber-forming part of the tube 22. This makes available a large discharge surface area which is accessible to the air flow.

Located beneath the stop collar 60, on the sleeve-like continuation 64, is an encircling groove 65 which is open in the direction of the outer annular wall 5; this can accommodate a sealing body.

A brief outline of the functioning is as follows: the user unscrews the closure cap 57 in order to bring about the ready-to-dispense position (FIG. 5). The dosing-chamber-forming parts here are drawn in the direction of the supply chamber 2 in the housing 3 by the claws 56 and the driver protuberances 51. This results in the claws 56 being uncoupled from the driver protrusions 47. The dosing-chamber wall 35, formed by the tube 22, is moved with scooping action through the powder supply, to be precise carrying along, in the process, the dosing-chamber base 34, which forms a cup-like cavity, that is to say is set back. This results in the dosing chamber 16 projecting partially in relation to the supply-chamber ceiling 11 according to FIG. 5, while containing a precisely reproducible sub-quantity 1′ on account of the function of the abovedescribed scraper 31.

The ready-to-dispense position, which can be achieved manually by the user, with relatively moderate forces, by the screwing-action capability, is achieved automatically.

All that is then required for the purpose of activating the dosing device D is for the user to produce a suction air stream S corresponding to how much needs to be inhaled. It is necessary to supply a deliberate suction air stream S; merely shallow breathing in is not sufficient and, in addition, would not result in the pulverulent substance successfully reaching the target area. It is only a sucking action which overcomes a threshold value which results in the pressure-exerting base 37 being actuated and, accordingly, in the sub-quantity 1′ being transferred into the suction air stream S. Only this achieves suction-air-controlled displacement of a sub-region of the dosing chamber 16 for exposing the sub-quantity 1′ to the suction air during the sucking action which exceeds the threshold value. The dosing-chamber volume empties.

The mouth aperture 9 is connected to the atmosphere via a suction channel 12, the interior 13, the through-openings 15, openings 46 the guide slot 52 and the longitudinal slots 48 and 55, by way of the gap 6. In the basic position, there is a relatively small throughflow cross-section in the region of the pressure-exerting base 37. This exerts a certain throttling action. It is only in the presence of a greater negative pressure on the upper side of the pressure-exerting base 37 that the threshold value is overcome and the rest of the travel path y is thus, as it were, abruptly covered, this causing the negative-pressure-controlled dosing-chamber base 34, which is in the form of a displaceable sub-region, to be moved out relative to the dosing-chamber wall 35. Inflowing outside air gains access via the abovedescribed path, see FIG. 6.

Once application has been completed, the dosing device D is transferred again from this position into the basic position according to FIG. 1, simply by the closure cap 57 being replaced and screwed closed again. In this case, the abutment flank 62 draws the tube-carrying connecting part 23 in again and, finally, draws it back into the starting position according to FIG. 1. In this movement direction, the claw 56 also has a downward pushing action, by way of its downwardly oriented abutment flank 62, on the strip head 56 of the pressure-exerting base 37. Once the tube 22 has lowered, the scraper 31, which is correspondingly capable of returning, also moves back again into its starting position, in which it is ready for scraping action (see FIG. 1).

A not yet discharged sub-quantity 1′ can be moved back into the supply again from the position according to FIG. 5 when the dispenser is screwed closed.

All features disclosed are (in themselves) pertinent to the invention. The disclosure content of the associated/attached priority documents (copy of the prior application) is hereby also included in full in the disclosure of the application, also for the purpose of incorporating features of these documents in claims of the present application.

1. Dosing device (D) which can be activated by the suction air stream (S) of the user and is intended for dispensing from a supply chamber (2) reproducible sub-quantities (1′) of powder (1), in particular powdered medicament, which is to be inhaled by the user, it being the case that a sub-quantity (1′) located in a dosing chamber (16), which has a dosing-chamber base (34) and a dosing-chamber wall (35), in the first instance is to be moved manually into a ready-to-dispense position and then a sucking action which overcomes a threshold value activation takes place for transferring the sub-quantity (1′) into the suction air stream (S), characterized by a suction-air-controlled displacement of a sub-region of the dosing chamber (16) for exposing the sub-quantity (1′) to the suction air during the sucking action which exceeds the threshold value.

2. Dosing device according to claim 1 or in particular according thereto, characterized in that the displaceable sub-region is in the form of a negative-pressure-controlled dosing-chamber base (34) which can be raised relative to the dosing-chamber wall (35).

3. Dosing device according to one or more of the preceding claims or in particular according thereto, characterized in that the dosing-chamber wall (35) is in the form of a tube (22) which accommodates the movable dosing-chamber base (34) and can be moved with scooping action through the powder supply.

4. Dosing device according to one or more of the preceding claims or in particular according thereto, characterized in that the dosing chamber (16) is coupled to a closure cap (57) of the dosing device (D) and, in the course of the closure cap (57) being removed from a housing (3) of the dosing device (D), is moved with scooping action through the powder supply.

5. Dosing device according to one or more of the preceding claims or in particular according thereto, characterized in that the closure cap (57) is coupled in a releasable manner by means of clip-action claws (56) to a connecting part (23) which carries the dosing chamber (16).

6. Dosing device according to one or more of the preceding claims or in particular according thereto, characterized in that the dosing-chamber base (34), which can be moved relative to the dosing-chamber wall (35), is coupled to the tube (22) via driver protuberances (51).

7. Dosing device according to one or more of the preceding claims or in particular according thereto, characterized in that the dosing-chamber base (34) is acted upon by a spindle (36), that end of the latter which is directed away from the supply chamber (2) having a pressure-exerting base (37) actuated by suction air stream (S).

8. Dosing device according to one or more of the preceding claims or in particular according thereto, characterized in that the dosing-chamber base (34) can be adjusted relative to the spindle (36) in order to achieve different sub-quantities (1′).

9. Dosing device according to one or more of the preceding claims or in particular according thereto, characterized by a screwing-action adjusting capability of the dosing-chamber base (34) relative to the spindle (36).

10. Dosing device according to one or more of the preceding claims or in particular according thereto, characterized in that the supply chamber (2) contains a scraper (31) which interacts with the dosing chamber (16) as it passes through.

11. Dosing device according to one or more of the preceding claims or in particular according thereto, characterized in that the scraper (31) is in the form of a radial finger which can be pushed aside from the displacement path.

12. Dosing device according to one or more of the preceding claims or in particular according thereto, characterized in that a supply-chamber ceiling (11) is in the form of a membrane (25), which allows through-passage as a result of slitting, or the ceiling is assigned such a membrane.

13. Dosing device according to one or more of the preceding claims or in particular according thereto, characterized in that the length of a slit (26) is adapted to the dosing chamber (16) passing through it such that the exiting dosing chamber (16) is enclosed in a sealing manner by the membrane (25).

14. Dosing device according to one or more of the preceding claims or in particular according thereto, the dosing device (D) having a mouth aperture (9) which is located above the supply chamber (2) and around which the user is to place his/her mouth, characterized in that a flow divider (29) is disposed in the mouth aperture (9).

15. Dosing device according to one or more of the preceding claims or in particular according thereto, characterized in that the flow divider (29) is in the form of a central body which tapers cross-sectionally in the direction of the dosing chamber (16).

16. Dosing device according to one or more of the preceding claims or in particular according thereto, characterized in that the closure cap (57) is accommodated in the housing (3), which retains the dosing chamber (16), via a screw thread (63).
